(19) United States
(12) Patent Application Publication
(10) Pub. No.: US 2004/0137420 A1
(43) Pub. Date: Jul. 15, 2004

(54) Method for analysis of substances in tissue or in cells

(76) Inventors: Akikazu Yasuda, Osaka (JP); Yoshimi Yasuda, Osaka (JP); Terumi Nakajima, Tokyo (JP)

Correspondence Address: MANELLI DENISON & SELTER, 2000 M STREET NW SUITE 700, WASHINGTON, DC, 20036-3307, US

(21) Appl. No.: 10/220,345

(22) PCT Filed: Mar. 2, 2001
(86) PCT No.: PCT/JP01/01640

(30) Foreign Application Priority Data
Mar. 6, 2000 (JP) 2000-060754

(51) Int. Cl. C12Q 1/00; C12Q 1/68; B01D 59/44

(52) U.S. Cl. 435/4; 435/6; 250/282

(57) ABSTRACT

An improved method for directly identifying a chemical structure of a substance present in tissue or cells of organisms is disclosed. In particularly, the method for directly identifying a chemical structure of a substance present in tissue or cells of various kind of organisms comprises the following steps:

(1) a step irradiating a laser to a certain intracellular region of a sample tissue slice or a cell, and analyzing the generating mass ions to obtain mass spectrum of the substance present at the cite,

(2) a step analyzing the mass spectrum to obtain a mass profile of the substance existing at the intracellular region of the sample tissue slice or in cell, and

(3) a step determining the chemical structure of the substance corresponding a certain molecular weight appeared in the mass profile. The method of this invention is conducted by utilizing the combined techniques of matrix-assisted laser desorption/ionization time-of-flight mass spectrometry (MALDI-TOF MS), and on-line capillary reversed-phase HPLC/quadrupole orthogonal acceleration time-of-flight (Q-Tof)-MS, and molecular cloning, is disclosed.

### TECHNICAL FIELD

This invention relates to an improved method for directly identifying a chemical structure of a substance present in tissue or cells of organisms. In particularly, this invention relates to the method for directly identifying a chemical structure of a substance present in tissue or cells of various kind of organisms which comprises the following steps:

(1) a step irradiating a laser to a certain intracellular region of a sample tissue slice or a cell, and analyzing the generating mass ions to obtain mass spectrum of the substance present at the cite,

(2) a step analyzing the mass spectrum to obtain a mass profile of the substance existing at the intracellular region of the sample tissue slice or in cell, and

(3) a step determining the chemical structure of the substance corresponding a certain molecular weight appeared in the mass profile.

The method of this invention is conducted by utilizing the combined techniques of matrix-assisted laser desorption/ionization time-of-flight mass spectrometry (MALDI-TOF MS), and on-line capillary reversed-phase HPLC/quadrupole orthogonal acceleration time-of-flight (Q-Tof)-MS, and molecular cloning.

### BACKGROUND ART

Neuropeptides in the brain and the nervous system are involved in the integration of complex physiological processes and behaviors. Thus, the clarification of the relationships between neuropeptides and their activities is an important goal of crustacean physiology. To identify neuropeptides, bioassays have been essential tools in the purification procedures. In fact, crustacean neuropeptides, e.g., red-pigment concentrating hormone, pigment-dispersing hormone, hyperglycemic hormone, molt-inhibiting hormone, vitellogenesis-inhibiting hormone, mandibular organ-inhibiting hormone, RFamide related peptides, cardioactive peptides and orcokinin have been isolated using bioassays based on physiological effects such as light adaptation, blood glucose regulation, molt inhibition, heart regulation, and gut contraction (Fernlund and Josefsson, 1972; Fernlund, 1976; Mercier et al., 1971; Stangier et al., 1987; Stangier et al., 1992; Wainwright et al., 1996; see reviews: Keller, 1992; Yasuda and Naya, 1997). However, the purification with such bioassays includes at least two problems. First, relatively large amounts of starting material are needed to leave enough peptide for sequencing due to the consumption of material in bioassays at each purification step. Second, with the available standard bioassays, many novel neuropeptides may go undetected.

Recently, matrix-assisted laser desorption/ionization time-of flight mass spectrometry (MALDI-TOF MS) has become a powerful tool for the direct analysis of peptide profiles from small pieces of dissected tissues or even isolated single cells (Garden et al., 1996; De With et al., 1997; Garden et al., 1998; Redeker et al., 1998). In these experiments, it is possible to analyze very small spots of samples, and the resulting peptide fingerprinting shows the synthesis and expression of bioactive peptides. In crustaceans, two molecular forms of hyperglycemic hormone precursor related peptides have been successfully characterized by the MALDI-TOF MS approach using single cells from the crayfish, Orconectes limosus (Redeker et al., 1998).

On the other hand, on-line capillary reversed-phase high performance liquid chromatography with mass spectrometric detection (capillary HPLC/MS and capillary HPLC/MS/MS) is available in modern bioanalysis. This tool provides excellent information on the molecular masses of neuropeptides in brain extract, and MS/MS mode allows the peptides to be fragmented to yield product ions enabling their amino acid sequences to be deduced. In the present invention, the inventors used a mass spectrometry-based protocol for identification of novel neuropeptides from the brain of the crayfish Procambarus clarkii, in which combinatorial approaches by MALDI-TOF MS, molecular cloning and on-line capillary HPLC-MS/MS were employed.

### DISCLOSURE OF INVENTION

The inventors initially examined the direct MALDI-TOF MS analysis of various slices from the brain of the red swamp crayfish, Procambarus clarkii. During this work, we noticed that a unique peptide with a molecular mass of 1517 is present in the brain. This molecular weight was identical to that of orcokinin (NFDEIDRSGFGFN) which was originally isolated from the ventral nerve cord of the crayfish, Orconectes limosus (Stangier et al., 1992). The biological effect of the peptide is a potent contracting activity on the isolated hindgut of the crayfish. Additionally, three orcokinin related peptides have been isolated and sequenced, i.e., [Ser⁹], [Ala¹³], and [Val¹³]orcokinin, from the shore crab Carcinus maenas (Bungart et al., 1995a). The inventors also found peptides having molecular weights corresponding to [Ala¹³] and [Val¹³]orcokinin, together with orcokinin, in the direct MALDI-TOF MS data obtained from a single spot of the sliced brain. Therefore, the inventors first focused on the identification of orcokinin and its related peptides in the crayfish brain.

The inventors disclose here the characterization of orcokinin and its related peptides and the molecular cloning of two orcokinin precursor proteins in the brain of the red swamp crayfish. In addition, a new strategy for the identification of neuropeptides, without the use of bioassays, is proposed.

The inventors developed a strategy for the exploration of unknown substances in tissue or in cells of various organism. More specifically, the inventors succeed to a novel strategy for the exploration unknown peptides and identifying the chemical structure of the peptides in tissue or in cells of various organisms.

### BRIEF DESCRIPTION OF DRAWINGS

FIG. 1. Direct MALDI-TOF MS spectrum of a slice from the olfactory lobe of the red swamp crayfish brain. The x-axis shows the m/z, mass to charge ratio; the y-axis shows the intensity of the molecular ions.

FIG. 2. The nucleotide (AB029168) and deduced amino acid sequences of the corresponding preproorcokinin A from the red swamp crayfish. Orcokinin and its related peptide sequences are boldfaced. The paired basic amino acid residues are indicated by the boxed letters. Polyadenylylation signals are denoted by underlining.

FIG. 3. Schematic representation of preproorcokinins A and B. The putative, hydrophobic signal sequence in each precursor is illustrated as “S”. Orcokinin and its related neuropeptide sequences present on both precursors are labeled with the same patterns and numbers:. 1, FDAFTTGFGHS; 2, NFDEIDRSGFGFA ([Ala¹³]orcokinin); 3, NFDEIDRSGFGFN (orcokinin); 4, NFDEIDRSGFGFV ([Val¹³]orcokinin); 5, NFDEIDRTGFGFH ([Thr⁸, His¹³]orcokinin).

FIG. 4. Elution profile of an acid extract of red swamp crayfish brains on a Sephadex G-25 column (superfine, 1×400 mm) equilibrated with 0.05 N acetic acid. The flow rate was 20 μl/min. Orcokinin and its related peptides were concentrated in fraction 2 which is marked by shading.

FIG. 5. Capillary reversed-phase high-performance liquid chromatography of the Sephadex G-25 fraction 2 on a TSK gel ODS 120T column (0.25×100 mm, 5 μm). The elution was performed using a linear gradient of acetonitrile in 0.05% TFA from 10% to 70% for 60 min. The detail experiment is described in the text. Selected ion chromatograms of orcokinin gene-related peptides were obtained by monitoring doubly charged ions at m/z 791 for orcokinin, m/z 752 for [Val¹³]orcokinin, m/z 738 for (Ala¹³]orcokinin, m/z 778 for [Thr⁸, His¹³]orcokinin, m/z 594 for FDAFTTGFGHS, and m/z 686 for VYVPRYIANLY.

FIG. 6. Collision-induced dissociation MS/MS spectra of doubly charged ions at m/z 759, 752, 738, 778, 594, and 686 in capillary HPLC preparation from brain extract corresponding to FIG. 5. The amino acid sequences of the peptides were assigned from yₙ ions series (Biemann, 1992).

### DETAILED DESCRIPTION OF INVENTION

In particular, the inventors developed the exploration and/or identification of peptides brain peptides in the red swamp crayfish, Procambarus clarkii, utilizing the combined techniques of matrix-assisted laser desorption/ionization with time-of-flight mass spectrometry (MALDI-TOF MS), molecular cloning and on-line capillary reversed-phase HPLC/quadrupole orthogonal acceleration time-of-flight (Q-Tof)-MS. The inventors initially performed direct MALDI-TOF MS analysis with slices of the brain. The MS spectra from a slice of the olfactory lobe indicated that an orcokinin (NFDEIDRSGFGFN) occurs in this species. Subsequently, its occurrence was confirmed by molecular cloning of the cDNAs encoding the precursor protein of orcokinin. The deduced amino acid sequences indicated that there are two different types of preproorcokinins. Preproorcokinin A (251 residues long) contains not only seven copies of orcokinin but also two copies of NFDEIDRSGFGFV and one copy each of NFDEIDRSGFGFA, NFDEIDRTGFGFH and FDAFTTGFGHS. The former three peptides were previously isolated from other crayfish, Orconectes limosus and/or the shore crab, Carcinus maenas, and the latter two were novel. Preproorcokinin B (266) harbors one additional orcokinin. All sequences of the peptides are flanked by dibasic sequences which are the consensus signal for processing. Moreover, brain extract was subjected to Sephadex G-25 and, subsequently, to on-line capillary reversed-phase HPLC/Q-Tof MS analysis. From the LC-MS analysis, the molecular weights of orcokinin, NFDEIDRSGFGFV, NFDEIDRSGFGFA, NFDEIDRTGFGFH, and FDAFTTGFGHS were identified as the doubly charged ions at m/z 759.37, 751.92, 737.86, 778.90, and 593.78, respectively. In addition,.the sequences were assigned by the collision-induced dissociation spectra using the doubly charged ions in the LC-MS/MS analysis. These data suggested that orcokinin and its related peptides are especially abundant in the olfactory lobe and are synthesized and processed from the two types of preproorcokinins in the crayfish brain.

The new strategy for the exploration of peptides in tissue or in cells of various organisms such as

microorganisms, plants, animals and human. According to the aspects of this invention,

the developed strategy can be applied to the following embodiments of methods:

1. A method for directly identifying a chemical structure of a substance present in tissue or cells of organisms, which comprises the steps:

(1) a step irradiating a laser to a certain intracellular region of a sample tissue slice or a cell, and analyzing the generating mass ions to obtain mass spectrum of the substance present at the cite,

(2) a step analyzing the mass spectrum to obtain a mass profile of the substance existing at the intracellular region of the sample tissue slice or in cell, and

(3) a step determining the chemical structure of the substance corresponding a certain molecular weight appeared in the mass profile.

2. A method for directly identifying the chemical structure of a substance present in tissue or cells according to Embodiment 1, wherein steps (1) and (2) are conducted by MALDI-TOF MS, and step (3) is conducted by LC-MS/MS and/or a protein sequencer.

3. A method for directly identifying the chemical structure of a substance present in tissue or cells according to Embodiment 1 or 2, wherein after the step (2) obtaining a mass profile of the substance present in a spot of the tissue slice or the cell, and before the step (3) determining the chemical structure of the substance corresponding a certain molecular weight appeared in the mass profile, a step in which the substance is isolated and purified from an extract of the tissue or cells by the use of the molecular weight of the substance as a marker, is added.

4. A method for directly identifying the chemical structure of a substance present in tissue or cells according to Embodiment 1, 2 or 3, wherein when the chemical structure of the substance is unknown, said structure is determined.

5. A method for directly identifying the chemical structure of a substance present in tissue or cells according to Embodiment 4, wherein when the existence of a substance having a specific biological activity is predicted in tissue or a cell but the chemical structure of the substance is unknown, the chemical structure is predicted according to Embodiments 1-3 and a substance having the predicted structure is synthesized and the chemical structure of the substance is confirmed by investigating the biological activity of the synthesized substance.

6. A method for identifying the chemical structure of an unknown substance present in tissue or cells of organism by conducting a method of one of Embodiments 1-3.

7. A method according to any one of Embodiments 1-6, wherein the amino acid sequence is determined when the substance is a protein or peptide.

8. A method for determining the amino acid sequence of a protein, a peptide or their precursor according to Embodiment 7, which comprises steps:

(1) chemically synthesizing a DNA having a base sequence corresponding to partial amino acid sequence of the substance,

(2) constructing a cDNA library from the sample tissue slice or cell,

(3) cloning a gene coding for the protein, peptide or their precursor by screening the cDNA library constructed in (2) with use of the synthesized DNA in (1) as a probe, and

(4) determining the base sequence of the cloned cDNA to deduce the amino acid sequence.

9. A method according to Embodiment 7 or 8, wherein a site of a modified amino acid residue or a modified amino acid in a modified peptide or a modified protein is determined when the substance is a modified peptide or a modified protein.

10. A method according to Embodiment 1, 2 or 3, wherein the method is directed to determine the site of the substance in the cell.

11. A method according to Embodiment 7, 8 or 9, wherein a site of the presence of a peptide, a protein, a modified peptide, a modified protein or their precursor in the cell is determined.

12. A method according to anyone of Embodiments 1-11 wherein the site of the presence of the substance to be detected is in a vesicle in the cell.

13. A method according to Embodiment 12 wherein said vesicle in the cell is secretory vesicle.

### EXAMPLES AND EXPERIMENTS

(Materials and Methods)

(MALDI-TOF MS)

The red swamp crayfish, Procambarus clarkii, was commercially obtained in Japan. The brain was dissected and immediately frozen followed by cutting with a razor blade. A small slice was then placed onto a MALDI sample plate. The matrix of α-cyano 4-hydroxycinnamic acid (α-CHCA) was saturated in a solution of acetonitrile/water 50:50 (v/v), containing 0.1% trifluoroacetic acid (TFA). To remove excess salts present in the sample, matrix rinsing was repeated three times (Garden et al., 1996). The fresh matrix solution was added to the sample and dried. MALDI-TOF mass spectra were acquired using a Voyager Elite MALDI-TOF mass spectrometer (Perceptive Biosystems, Framingham, Mass., USA) equipped with a delayed extraction source and 337 nm pulsed nitrogen laser. The acceleration voltage for the linear mode was 20 kV with the grid voltage set at 91%. The delay time was 50 ns. External calibration was performed using the insulin B-chain and the protonated matrix dimer ion.

(RNA Preparation and cDNA Library Construction)

Total RNA from the brain and the suboesophageal ganglia (20 animals) was prepared using TRIzol Reagent (Total RNA Isolation Reagent, GIBCO BRL). Poly(A)⁺RNA was purified by the batch elution method from Oligotex-™dT30 (Roche, Japan). cDNA was prepared using a λZAP Express cDNA Synthesis kit (Stratagene, Calif.). The cDNA was ligated into the λZAP Express arms and packaged with a packaging mixture (Gigapack III Gold Packaging Extract, Stratagene).

(Screening of the cDNA Library)

The cDNA library was screened with 192-fold degenerated 20-mer oligonucleotides, [5′-AA(C/T)TT(C/T)GA(C/T)GA(A/G)AT(A/C/T)GA(C/T)(A/C)G-3′]. They were synthesized according to the N-terminal amino acid sequence of orcokinin (Asn-Phe-Asp-Glu-Ile-Asp-Arg). Labeling, hybridization and detection were carried out according to the protocol of the ECL 3′-oligolabelling and detection systems (Amersham, UK) except for the composition of the wash solution. Briefly, the transferred Hybond-N⁺ membranes (Amersham, UK) were prehybridized for 1 h at 42° C. and then hybridized with labeled probes overnight at 42° C. in the hybridization solution. Washings were performed three times at 49° C. for 30 min with wash solutions containing tetramethylammonium chloride as described by Jacobs et al. (1988). Plasmids were rescued from the positive plaques by in vivo excision. The obtained clones were successively analyzed by DNA sequencing (ABI Prism 310, Perkin-Elmer, Calif.).

(Capillary Reversed-Phase HPLC/Q-Tof MS)

The brains were heated at 80° C. in 100 μl of 0.1 N hydrochloric acid for 3 min, rapidly cooled to 4° C., and homogenized. After centrifugation, the supernatant was lyophilized. The extract was subjected to gel filtration on a Sephadex G-25 column (superfine, 400 mm×1 mm i.d.) equilibrated with 0.05 M acetic acid. The mid fraction was subjected to capillary reversed-phase high-performance liquid chromatography (HPLC) using a PEEK tube (100 mm×0.25 mm i.d.) packed with in-house TSK gel ODS 120T (Tosoh, 5 μm particle size) in the Hewlett-Packard HP1100 liquid chromatography system with a linear gradient of acetonitrile containing 0.05% TFA. The flow from the pump (100 μl/min) was split by a T-connector and the flow toward the HPLC column was adjusted to 2 μl/min. Sample (1 μl, one brain equivalent) was applied via a Valco injection valve placed between the T-connector and the capillary column. The eluate was monitored at 220 nm using a UV detector equipped with a U-shaped cell (LC Packings, model UZ-HP11-CAP). The outlet of the UV detector was connected to the electrospray interface of the mass spectrometer. The mass spectrum of the eluate was detected using a Q-Tof mass spectrometer (Micromass, Manchester, UK). Typically, 2800 V was applied to the spraying capillary and 50 V to the sample cone. The source temperature was kept at 50° C. The range of the total-ion current was m/z 100 to 2000. For the LC/MS/MS experiment, the mass spectrometer was set to automatic data-dependent MS to MS/MS switching when the intensity of the precursor ion increased to over 20 counts/s.

The collision energy was 30 V for doubly charged precursor ions.

(Results)

(MALDI-TOF MS)

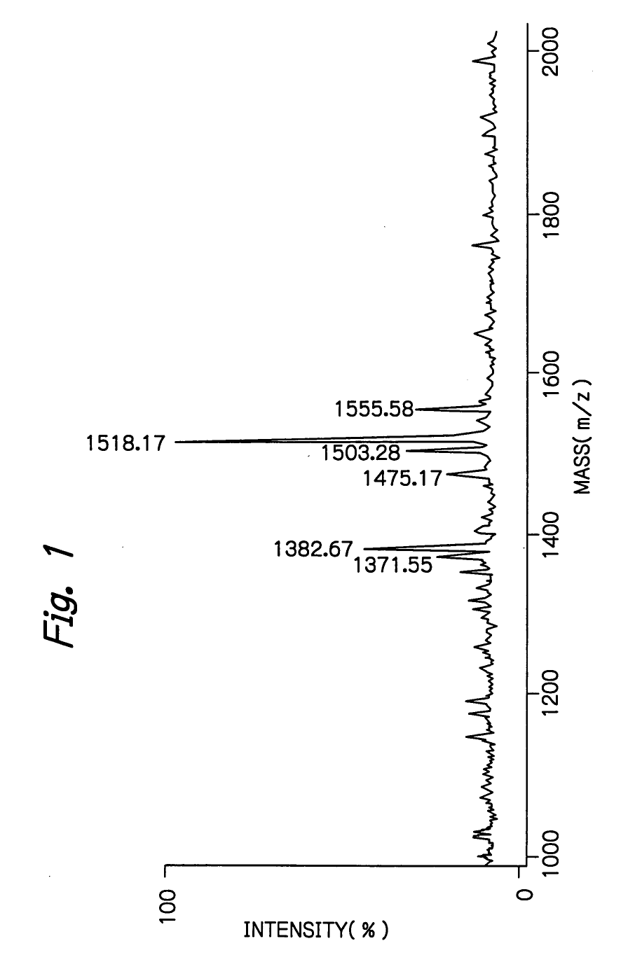

Direct application of the MALDI-TOF-MS to slices of crayfish brain revealed that a peptide at mass m/z 1518 was observed at several points. Among them, the spectra of a slice from the olfactory lobe was specifically dominated by the intensity of the m/z 1518.17 peak, as shown in FIG. 1. In addition, five peaks observed at mass m/z 1371.55, 1382.67, 1475.17, 1503.28, and 1555.58 were detected together in the olfactory lobe. A peptide search with the molecular weight in the known crustacean peptides revealed orcokinin with a calculated average mass at 1517.6, [Val¹³] orcokinin with the mass at 1502.6 and [Alar¹³] orcokinin with the mass at 1474.5, as summarized in Table 1. Therefore, this information implied that orcokinin family peptides occur in the brain of the red swamp crayfish. After the cloning of preproorcokinin and the capillary HPLC/MS/MS analysis described below, peaks at mass m/z 1371.55 and 1555.58 were identified as a peptide of VYVPRYIANLY and [Thr⁸, His¹³]orcokinin, respectively. A peak at mass m/z 1382.67 seemed to be from the other unknown neuropeptide.

(Cloning of cDNAs Encoding the Orcokinin Precursor Proteins)

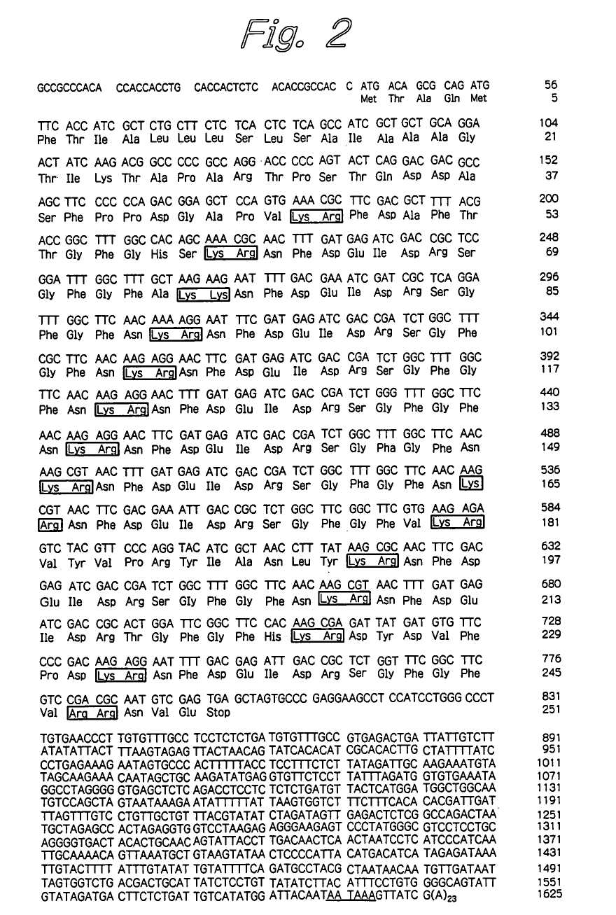

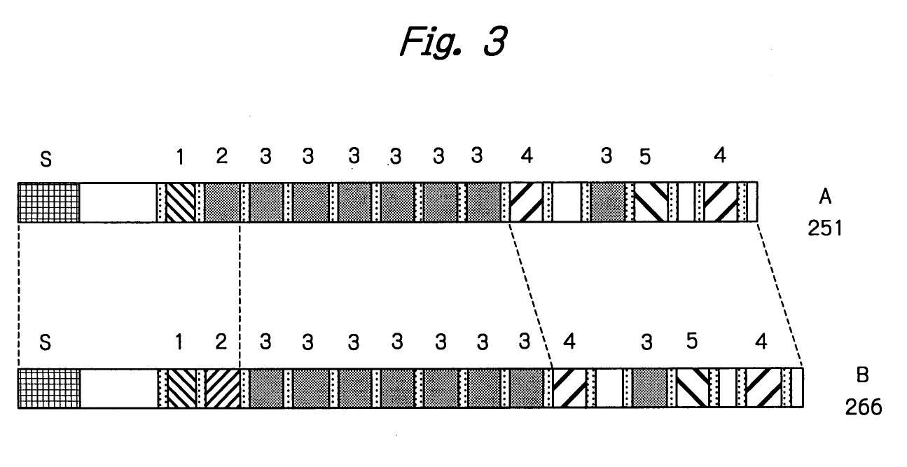

Further confirmation of the existence of orcokinins in the red swamp crayfish was performed by molecular biological studies. A λZAP Express cDNA library from the brain and the suboesophageal ganglia of the crayfish was constructed, and approximately 5×10⁵ recombinant phages were screened with the probes synthesized from 192-fold degenerated oligonucleotides according to the sequence of NFDEIDR. Fourteen positive clones were isolated and found to contain inserts ranging from about 1.1 to 1.8 kb. They all contain one or two AATAAA consensus sequences for the mRNA polyadenylylation in the 3′-UTR and also a poly A tail of 20-100 nucleotides at the 3′ end. The sequence analysis indicated that they encode two different types of orcokinin precursor proteins. The two representative nucleotide sequences of the clones have been submitted to the GenBank/EMBL/DDBJ Data Bank with accession no. AB029168 (1602 bp) and AB029169 (1240 bp). AB029168 and AB029169 had the longest open-reading frames of 753 bp and 798 bp, with the 41 bp and 74 bp of 5′-UTRs, and the 808 bp and 368 bp of 3′-UTRs except for the poly(A) tail, respectively. The corresponding deduced amino acid sequences of AB029168 (251 residues long) and AB029169 (266 residues long) were named preproorcokinin A and B, respectively. FIG. 2 shows the nucleotide sequence of AB029168 and the deduced amino acid sequence of the preproorcokinin A. The first Met is followed by a stretch of about 20 uncharged amino acids which was identified as the most hydrophobic region (data not shown) and as a signal sequence. Preproorcokinin A contains not only seven copies of orcokinin but also four kinds of other orcokinin-like peptides. That is, two copies of NFDEIDRSGFGFV ([Val¹³]orcokinin) and one copy each of NFDEIDRSGFGFA ([Ala¹³]orcokinin), NFDEIDRTGFGFH ([Thr⁸, His¹³]orcokinin) and FDAFTTGFGHS. The last two are novel orcokinin-like peptides. On the other hand, preproorcokinin B is the same as that of preproorcokinin A except it harbors one additional orcokinin sequence (FIG. 3). All sequences of the orcokinin and orcokinin-like peptides in the preproorcokinins were flanked by the dibasic sequences of KR, KK or RR at the N- and C-terminal ends in the precursor proteins.

(Capillary Reversed-Phase HPLC/Q-Tof MS)

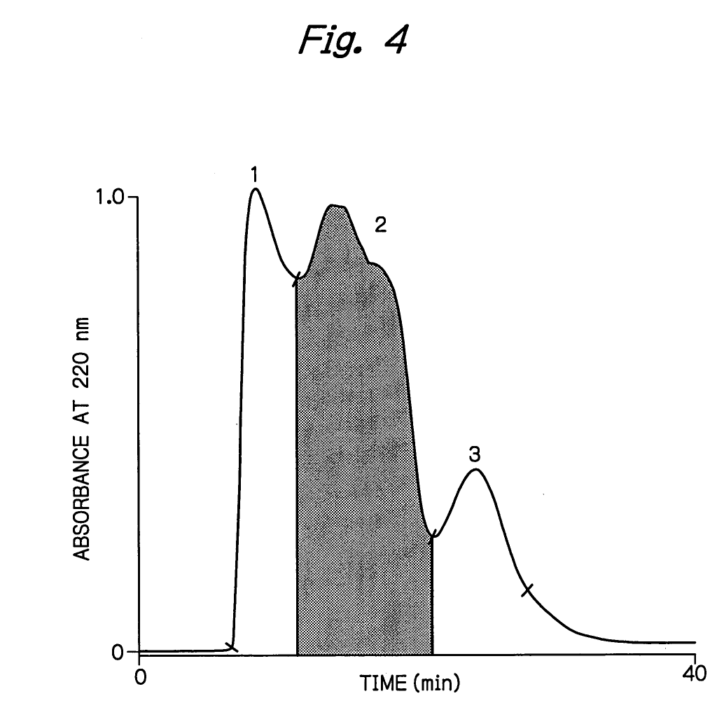

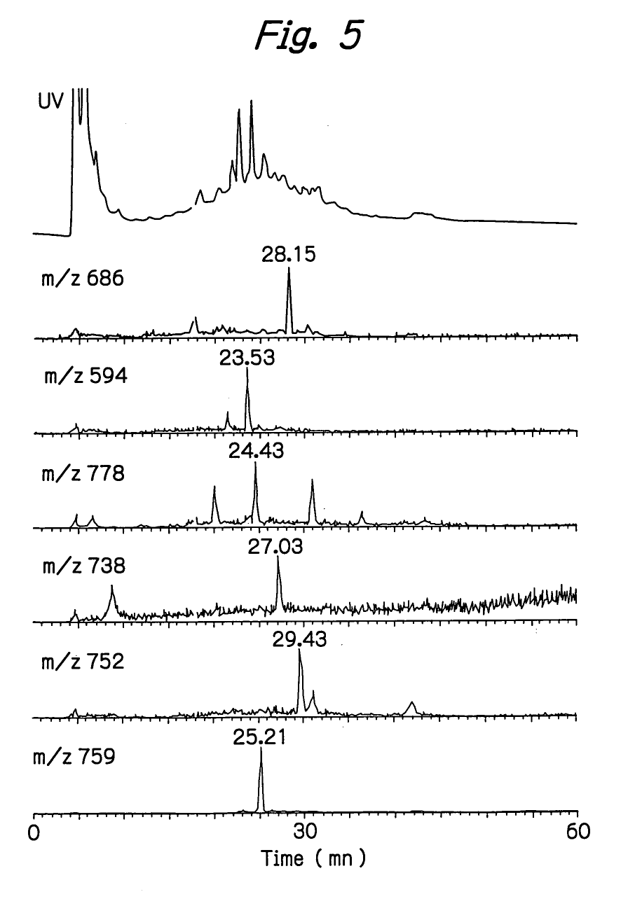

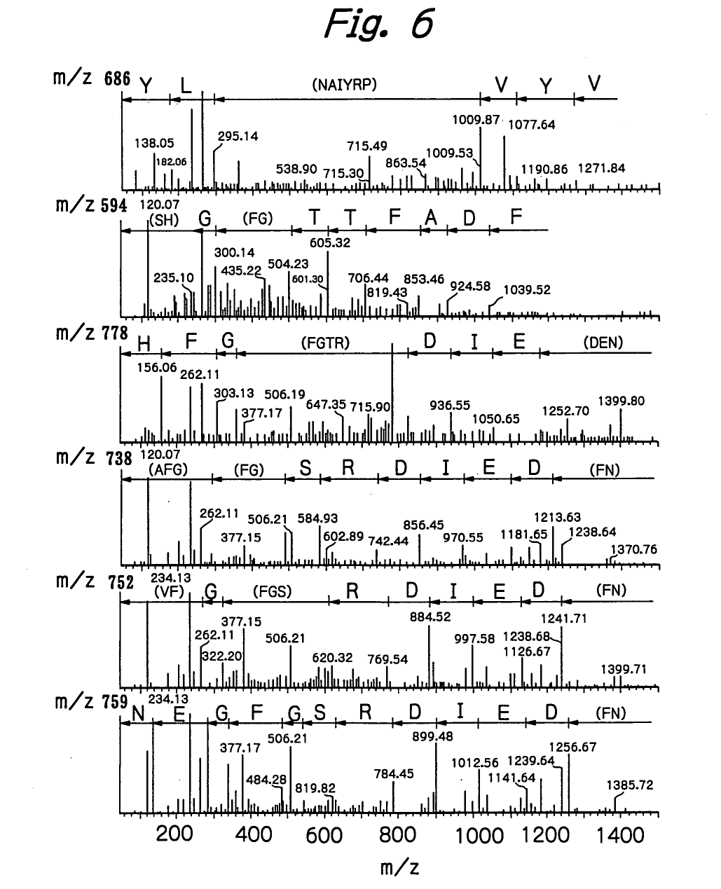

FIG. 4 shows the capillary gel filtration of the HCl extract of the crayfish brains (2 animals) on a Sephadex G-25 column. The second fraction contained a peptide at m/z 1518, -and was further subjected into on-lined capillary reversed-phase HPLC/Q-Tof MS. FIG. 5 shows the elution profile for one animal equivalent of the second fraction on a capillary TSK gel ODS 120T column. For identification of orcokinin and its related peptides, the calculated molecular weights are summarized in Table 1. The acquired LC-MS data was selectively monitored by the doubly charged ions of the peptides, as shown in FIG. 5. The doubly charged ions were observed at m/z 759.37, 751.92, 737.86, 777.90, and 593.78 in the chromatogram, and then transformed into masses of 1516.74, 1501.84, 1473.72, 1553.80, and 1185.56 that corresponded to the monoisotopic mass for orcokinin, [Val¹³]orcokinin, [Ala¹³]orcokinin, (Thr⁸, His¹³]orcokinin, and FDAFTTGFGHS, respectively. In addition, a doubly charged ion of m/z 685.91 present in the chromatogram, a transformed mass of 1369.82, that corresponded to the VYVPRYIANLY sequence in the proorcokinins. FIG. 6 shows the LC-MS/MS spectra of the orcokinin gene-related peptides on the Q-Tof MS. These collision-induced dissociation spectra, generated from the doubly charged ions at m/z 759, 752, 738, 778, 593 and 686, indicated fragmentation patterns for the sequences of orcokinin, [Val¹³]orcokinin, [Ala¹³]orcokinin, [Thr⁸, His¹³]orcokinin, FDAFTTGFGHS, and VYVPRYIANLY, as illustrated in FIG. 6.

(Discussion)

This is the first report on the application of the combined approaches of MALDI-TOF MS, molecular cloning and on-lined capillary reversed-phase HPLC/Q-Tof MS in crustacean neuropeptide studies. One of advantages in this strategy is that the neuropeptides are identified without referring to a given bioassay. Thus, this method could reduce the number of samples and time of the experiments which are needed to isolate the neuropeptides. First, the direct MALDI-TOF analysis of the peptides profiles of the olfactory lobe in the brain revealed a set of peptides specifically contained at [M+H]⁺ of m/z 1371.55, 1382.67, 1475.17, 1503.28, 1518.17, and 1555.58. In the present case, the molecular weight of orcokinin, [Val¹³], and [Ala¹³]orcokinin, which were previously isolated from the shore crab (Bungart et al., 1995a) and/or other crayfish (Burdzik et al., 1993; Stangier et al., 1992), was matched in the mass profile. Thus, the inventors examined the cloning of the orcokinin precursor. On the basis of the cloned pro-orcokinin structure, molecular weight and sequence of putative mature peptides were summarized. Finally, capillary reversed-phase HPLC/MS/MS was used to elucidate the occurrence of the mature peptides generated from the pro-orcokinins and to determine the structures of these peptides.

After cloning, reconsidering the peptide profile obtained from the direct MALDI-TOF MS analysis, besides orcokinin, [Ala¹³] and [Val¹³]orcokinin, a peak at mass m/z 1371.55 was identified as VYVPRYIANLY which is one of the linked peptides, and m/z 1555.58 which was [Thr⁸, His¹³]orcokinin. A short peptide of FDAFTTGFGHS could not be detected in the MS analysis (FIG. 1), because the intensity of the peptide ions in the MALDI-TOF MS is not necessarily quantitative to the peptide content in the sample mixture. However, the application of the direct MALDI-TOF MS analysis to the slice of brain allows preliminary and fast screening of many cells in neuropeptide studies, because the spectra obtained from a single spot convinces us of the profile for a specific processing of precursor proteins.

Capillary reversed-phase HPLC/Q-Tof MS is a powerful tool for the characterization of peptides and proteins. The advantages of a capillary column with respect to enhanced sensitivity of the peptide detection are that the LC-MS can be used to identify endogenous neuropeptides at the femtomole level and computer techniques offer a higher level of detection specificity, as shown in FIG. 5. Thus, the brain extract of one animal equivalent is quite adequate for the identification and sequencing of the neuropeptides. Consequently, on the basis of our research program, the inventors propose the following as a new standard procedure for the characterization of brain peptides in crustaceans. The first step is the site-specific molecular mass profiling of neurosecretory granules by direct MALDI-TOF MS analysis. The second step is purification of a given secretory substance by monitoring its molecular mass and then sequencing it. Both the LC-MS/MS and/or protein sequencer can be used for the structural determination. The third step is molecular cloning for the precursor of a given peptide based on the amino acid sequence of the peptide. The final step is capillary reversed-phase HPLC/Q-Tof analysis with the brain extract. The extract includes secretory substances that are fractionated into individual components by HPLC and then detected by the MS mode. The mass of each one can be selected from original LC-MS data by computer processing, and their sequences are determined by the MS/MS mode. These procedures will provide an excellent means for micro-characterization of novel neuropeptides in the crayfish brain, including site of the neuropeptides expression, precursor structure, and routes of processing.

This is also the first report on the cloning of the precursor proteins of orcokinin-related peptides. In the precursor protein, orcokinin-related peptides tandemly emerged and were flanked by dibasic residues. Its feature is similar to those of the precursor proteins of the small neuropeptides from molluscs, insects and/or nematodes, such as achatin-I (Satake et al., 1999), fulicin (Yasuda-Kamatani et al., 1995), myomodulin (Lopez et al., 1993) and FMRFamide (e.g. Linacre et al., 1990; Nambu et al., 1988; Rosoff et al., 1992). In crustaceans, the cDNAs of the precursor protein of the longer neuropeptides like hyperglycemic hormone and molt-inhibiting hormone have been clarified from several species (e.g. Weidemann et al., 1989; Tensen et al., 1991; Klein et al., 1993), however, these proteins contain only a single copy of the neuropeptide. To our knowledge, the orcokinin precursor protein from the red swamp crayfish is the first example in crustaceans, which encodes multiple copies of neuropeptides. Moreover, molecular cloning of cDNAs revealed that there are at least two kinds of orcokinin precursor proteins in the red swamp crayfish. They are the same except for only one additional copy of orcokinin. It is unclear why such similar proteins should be processed. However, our preliminary results obtained by reverse transcriptase-PCR indicated the existence of two types of transcripts in the brain (data not shown). In order to clarify whether the transcripts might be generated by alternative splicing from a single gene or derived from two or more different genes, Southern blotting and cloning of the genomic clones should be performed. Furthermore, Bungart et al. (1995a) suggested that orcokinin-related peptides might establish a novel neuropeptide family of orcokinins in crustaceans and possibly arthropods. The occurrence of orcokinin analogues in the crayfish brain supported this idea and the information about the nucleotide sequence of the precursor protein should prove to be a useful tool for the studies on the distribution of the orcokinin family among arthropods, molluscs and other invertebrates.

Concerning the biological activities, the short analog is the most interesting one. Except for the peptide of FDAFTTGFGHS, the five peptides identified from the three species are structurally very similar to each other and the differences are limited to positions 8, 9, and 13 (Table 1).

TABLE 1
Comparison of Amino Acid Sequence and MS of Orcokinin Gene-Related Peptides

| | | Calculated | Calculated | |
|---|---|---|---|---|
| Sequence | Name | average MS | monoisotopic MS | Species |
| 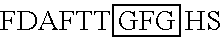 | | 1186.2 | 1185.509 | P.clarkii |
| 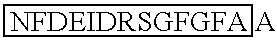 | [Ala¹³]orcokinin | 1474.5 | 1473.652 | P.clarkii, C.maenas^{a} |
| 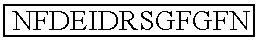 | orcokinin | 1517.6 | 1516.658 | P.clarkii, O.limosus^{b} |
| 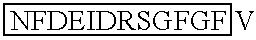 | [Val¹³ ]orcokinin | 1502.6 | 1501.684 | P.clarkii, C.maenas^{a}O.limosus^{c} |
| 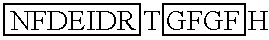 | [Thr⁸, His¹³]orcokinin | 1554.6 | 1553.690 | P.clarkii |
| 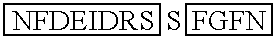 | [Ser⁹] orcokinin | 1547.6 | 1546.669 | C.maenas^{a} |

^{a}Taken from Bungart et al., (1995a).
^{b}Taken from Stangier. at al., (1992),
^{c}Taken from Burdzik at al., (1993)

Bungart et al. (1995b) reported that changes in orcokinin at the C-terminus interfere less with the activity on the hindgut than the N-terminal modifications. It could be then speculated that one of the novel peptides, [Thr⁸, His¹³]orcokinin, possesses such activity on the crayfish hindgut like the other orcokinin analogs. On the contrary, the structure of other novel peptide, FDAFTTGFGHS, is quite different from those of orcokinin and its related peptides. Therefore, its biological activity should be examined on some possible targets, such as the heart or neurons as well as on the hindgut. On the other hand, it is considered that the olfactory lobe receives its total input from the chemoreceptors on the first antenna, since axons from the olfactory receptor neurons on the antennule project exclusively into the olfactory glomeruli in the neurophile of the olfactory lobe in the crayfish brain (Sandeman et al., 1992). However, the physiological role of orcokinin and its gene-related peptides in the brain is not yet defined at this time. The finding and elucidation of the role of brain peptides, not to mention orcokinin, will become a further interesting project in crustacean physiology.

Industrial Applicability

In conclusion, orcokinin and its gene-related peptides from the crayfish brain have been characterized by monitoring with physicochemical criterion as alternatives to bioassay. In practice, it is advantageous to use MALDI-TOF MS, capillary reversed-phase HPLC/MS, and molecular cloning for the identification of brain peptides. The present investigation has also provided the first evidence for the structural organizations of the orcokinin precursor and its gene-related peptides produced by specific processing in the brain.

(References)

Biemann, K. (1992). Mass spectrometry of proteins and peptides. Annu. Rev. Biochem. 61, 977-1010.

Bungart, D., Hilbich, C., Dircksen, H., and Keller, R. (1995a). Occurrence of analogues of the myotropic neuropeptide orcokinin in the shore crab, Carcinus maenas: Evidence for a novel neuropeptide family. Peptides 16, 67-72.

Bungart, D., Kegel, G., Burdzik, S., and Keller, R. (1995b). Structure-activity relationships of the crustacean myotropic neuropeptide orcokinin. Peptides 16, 199-204.

Burdzik, S., Sauter, A., Stangier, J., and Keller, R. (1993). A novel orcokinin-related myotropic peptide from the hindgut of the crayfish, Orconectes limosus. In Proceedings of the 21th Gottingen Neurobiology Conference (Elsner, N. and Heisenberg, M., eds.) pp. 598, Stuttgart, Thieme.

De With, N. D., Li, K. W., Jimenez, C. R., Vonk, K., Dreisewerd, K., Hillenkamp, F., Karas, M., and Geraerts, W. P. M. (1997). Intracellular degradation of C-peptides in molluscan neurons producing insulin-related hormones. Peptides 18, 765-770.

Fernlund, P. and Josefsson, L. (1972). Crustacean color-change hormone: Amino acid sequence and chemical synthesis. Science 177, 173-174.

Fernlund, P. (1972). Structure of a light-adapting hormone from the shrimp, Pandalus borealis. Biochem. Biophys. Acta 439, 17-25.

Garden, R. W., Moroz, L. L., Moroz, T. P., Shippy, S. A., and Sweeler, J. V. (1996). Excess salt removal with matrix rinsing: direct peptide profiling of neurons from marine invertebrates using matrix-assisted laser desorption/ionization time-of-flight mass spectrometry. J. Mass. Spectrom. 31, 1126-1130.

Garden, R. W., Shippy, S. A., Li, L., Moroz, T. P., and Sweeler, J. V. (1998). Proteolytic processing of the Aplysia egg-laying hormone prohormone. Proc. Natl. Acad. Sci. U S A 95, 3972-3977.

Keller, R. (1992). Crustacean neuropeptides: Structures, functions and comparative aspects. Experientia 48, 439-448.

Klein, J. M., Mangerich, S., de Kleijn, D. P. V., Keller, R., and Weidemann, W. (1993). Molecular cloning of crustacean molt-inhibiting hormone (MIH) precursor. FEBS Lett. 334, 139-142.

Linacre, A., Kellett, E., Saunders, S., Bright, K., Benjamin, P. R., and Burke, J. F. (1990). Cardioactive neuropeptide Phe-Met-Arg-Phe-NH₂ (FMRFamide) and novel related peptides are encoded in multiple copies by a single gene in the snail Lymnaea stagnalis. J. Neurosci. 10, 412-419.

Lopez, V., Wickham, L., and Desgroseillers, L. (1993). Molecular cloning of myomodulin cDNA, a neuropeptide precursor gene expressed in neuron L10 of Aplysia californica. DNA and Cell Biology 12, 53-61.

Mercier, A. J., Orchard, I., Skerrett, M., and Te Brugge, V. (1971). FMRFamide related peptides from crayfish pericardial organs Soc. Neurosci. Abstr. 85, 200.

Nambu, J. R., Murphy-Erdosh, C., Andrews, P. C., Feistner, G. J., and Scheller, R. H. (1988). Isolation and characterization of a Drosophila neuropeptide gene. Neuron 1, 55-61.

Redeker, V., Toullec, J.-Y., Vinh, J., Rossier, J., and Soyez, D. (1998). Combination of peptide profiling by matrix-assisted laser desorption/ionization time-of-flight mass spectrometry and immunodetection on single glands or cells. Anal. Chem. 70, 1805-1811.

Rosoff, M., Büglin, T., and Li, C. (1992). Alternatively spliced transcripts of the flp-1 gene encode distinct FMRFamide-like peptides in Caenorhabditis elegans. J. Neurosci. 12, 2356-2361.

Sandeman, D., Sandeman, R., Derby, C., and Schmidt, M. (1992). Morphology of the brain of crayfish, crabs, and spiny lobsters: a common nomenclature for homologous structures. Biol. Bull. 183, 304-326.

Satake, H., Yasuda-Kamatani Y., Takuwa, K., Nomoto, K., Minakata, H., Nagahama, T., Nakabayashi, K., and Matsushima, O. (1999). Characterization of a cDNA encoding a precursor polypeptide of a D-amino acid-containing peptide, achatin-I and localized expression of the achatin-I and fulicin genes. Eur. J. Biochem. 261, 130-136.

Stangier, J., Hilbich, C., Beyreuther, K., and Keller, R. (1987). Unusual cardioactive peptide (CCAP) from pericardial organs of the shore crab, Carcinus maenas. Proc. Natl. Acad. Sci. USA 84, 575-580.

Stangier, J., Hilbich, C., and Keller, R. (1992). Orcokinin: a novel myotropic peptide from the nervous system of the crayfish, Orconectes limosus. Peptides 13, 859-864.

Tensen, C. P., De Kleijin, D. P. V., and Van Herp, F. (1991). Cloning and sequence analysis of cDNA encoding two crustacean hyperglycemic hormones from the lobster, Homarus americanus. Eur. J. Biochem. 200, 103-106.

Wainwright, G, Webster, S. G., Wilkinson, M. C., Chung, J. S., and Rees, H. H. (1996). Structure and significance of mandibular organ-inhibiting hormone in the crab, Cancer pagurus. Involvement in multihormonal regulation of growth and reproduction. J. Biol. Chem. 271, 12749-12754.

Weidemann, W., Gromoll, J., and Keller, R. (1989). Cloning and sequence analysis of cDNA for precursor of a hyperglycemic hormone. FEBS Lett. 257, 31-34.

Yasuda, A. and Naya, Y. (1997). Hormones in the red swamp crayfish. In Studies in Natural Products Chemistry Vol.19, Structure and Chemistry (Part E) (Atta-Ur-Rahman, Ed.) pp.627-687, Elsevier Science Publishers B.V., Amsterdam.

Yasuda-Kamatani, Y., Nakamura, M., Minakata, H., Nomoto, K., and Sakiyama, F. (1995). A novel cDNA sequence encoding the precursor of the D-amino acid-containing neuropeptide fulicin and multiple α-amidated neuropeptides from Achatina fulica. J. Neurochem. 64, 2248-2255.

All patents, patent application and publications cited above are incorporated herein by reference in their entirety.

The present invention is not limited in scope by the specific embodiments described, which are intended as single illustrations of individual aspects of the invention. Indeed, various modifications of the invention, in addition to those shown and described herein, will become apparent to those skilled in the art from the foregoing description. Such modifications are intended to fall within the scope of the appended claims.

1. A method for directly identifying a chemical structure of a substance present in tissue or cells of organisms, which comprises the steps:
(1) a step irradiating a laser to a certain intracellular region of a sample tissue slice or a cell, and analyzing the generating mass ions to obtain mass spectrum of the substance present at the cite,
(2) a step analyzing the mass spectrum to obtain a mass profile of the substance existing at the intracellular region of the sample tissue slice or in cell, and
(3) a step determining the chemical structure of the substance corresponding a certain molecular weight appeared in the mass profile.

2. A method for directly identifying the chemical structure of a substance present in tissue or cells according to claim 1, wherein steps (1) and (2) are conducted by MALDI-TOF MS, and step (3) is conducted by LC-MS/MS and/or a protein sequencer.

3. A method for directly identifying the chemical structure of a substance present in tissue or cells according to claim 1 or 2, wherein after the step (2) obtaining a mass profile of the substance present in an intracellular region of the tissue slice or the cell, and before the step (3) determining the chemical structure of the substance corresponding a certain molecular weight appeared in the mass profile, a step in which the substance is isolated and purified from an extract of the tissue or cells by the use of the molecular weight of the substance as a marker, is added.

4. A method for directly identifying the chemical structure of a substance present in tissue or cells according to claim 1, 2 or 3, wherein when the chemical structure of the substance is unknown, said structure is determined.

5. A method for directly identifying the chemical structure of a substance present in tissue or cells according to claim 4, wherein when the existence of a substance having a specific biological activity is predicted in tissue or a cell but the chemical structure of the substance is unknown, the chemical structure is predicted according to claims 1-3 and a substance having the predicted structure is synthesized and the chemical structure of the substance is confirmed by investigating the biological activity of the synthesized substance.

6. A method for identifying the chemical structure of an unknown substance present in tissue or cells of organism by conducting a method of one of claims 1-3.

7. A method according to any one of claims 1-6, wherein the amino acid sequence is determined when the substance is a protein or peptide.

8. A method for determining the amino acid sequence of a protein, a peptide or their precursor according to claim 7, which comprises steps:
(1) chemically synthesizing a DNA having a base sequence corresponding to partial amino acid sequence of the substance,
(2) constructing a cDNA library from the sample tissue slice or cell,
(3) cloning a gene coding for the protein, peptide or their precursor by screening the cDNA library constructed in (2) with use of the synthesized DNA in (1) as a probe, and
(4) determining the base sequence of the cloned cDNA to deduce the amino acid sequence.

9. A method according to claim 7 or 8, wherein a site of a modified amino acid residue or a modified amino acid in a modified peptide or a modified protein is determined when the substance is a modified peptide or a modified protein.

10. A method according to claim 1, 2 or 3, wherein the method is directed to determine the site of the substance in the cell.

11. A method according to claim 7, 8 or 9, wherein a site of the presence of a peptide, a protein, a modified peptide, a modified protein or their precursor in the cell is determined.

12. A method according to anyone of claims 1-11 wherein the site of the presence of the substance to be detected is in a vesicle in the cell.

13. A method according to claim 12 wherein said vesicle in the cell is secretory vesicle.
